(19) United States
(12) Patent Application Publication
(10) Pub. No.: US 2015/0257740 A1
(43) Pub. Date: Sep. 17, 2015

(54) BODY FAT DIAGNOSTIC APPARATUS

(71) Applicants: OSAKA PREFECTURE UNIVERISTY PUBLIC CORPORATION, Osaka (JP); PUBLIC UNIVERSITY CORPORATION OSAKA CITY UNIVERSITY, Osaka (JP); ADVANTEST CORPORATION, Tokyo (JP)

(72) Inventors: Hiromichi HORINAKA, Osaka (JP); Hiroyasu MORIKAWA, Osaka (JP); Taiichiro IDA, Tokyo (JP)

(21) Appl. No.: 14/634,519

(22) Filed: Feb. 27, 2015

(30) Foreign Application Priority Data
Mar. 14, 2014 (JP) 2014-052595

(51) Int. Cl. A61B 8/08; A61B 8/00; A61B 8/14; A61B 5/00

(52) U.S. Cl. CPC A61B 8/5223; A61B 5/4872; A61B 8/08; A61B 8/5207; A61B 8/4483; A61B 8/14

(57) ABSTRACT

A body fat diagnostic apparatus with which fat can be diagnosed safely is provided even in the case where a deep portion of the living body is diagnosed or there are bone tissues outside the measurement region. The body fat diagnostic apparatus is formed of: a probe 2 for emitting ultrasonic waves both for applying heat and for diagnosis; and an ultrasonic wave velocity change analyzing unit 15 for calculating the change in the velocity of ultrasonic waves in the measurement region on the basis of the ultrasonic wave echo signals acquired from the region before and after the application of heat using the probe 2, so that fat is diagnosed on the basis of the calculated change in the velocity of ultrasonic waves.

### CROSS-REFERENCE TO RELATED APPLICATIONS

This application claims priority to Japanese Patent Application No. 2014-052595 filed Mar. 14, 2014, the subject matter of which is incorporated herein by reference in entirety.

### BACKGROUND OF THE INVENTION

1. Field of the Invention

The present invention relates to a body fat diagnostic apparatus for applying heat to a measurement region such as a region of interest (ROI) of a subject so as to detect a change in the velocity of ultrasonic waves before and after the application of heat to the region, and thus, for diagnosing fat tissues. In particular, the present invention relates to a body fat diagnostic apparatus that is appropriate for fat diagnosis in a deep portion of the body, more specifically, for diagnosing fat inside the ribcage such as in the liver.

2. Description of Related Art

As a diagnostic technology for diagnosing the state within the body, a sound wave measuring apparatus for applying heat to a subject through irradiation of thermal energy, such as light, electrical waves or, in some cases, ultrasonic waves, from the outside of the subject so as to measure a change in the velocity of ultrasonic waves before and after the application of heat, and thus, for measuring the characteristics of the temperature change or the energy absorbing properties in the heated portion has been disclosed (see Patent Document 1).

This document describes typical examples that use a light emitting mechanism. That is to say, an example where a subject is irradiated with light in all directions in such a manner that the energy density becomes as uniform as possible, and an example where the entire body of a subject is laterally irradiated with light from a light source of which the width is greater than that of the body of the subject are cited, and thus, it is disclosed that heat is applied widely to the entirety of the region of interest including its surroundings for measurement. Meanwhile, nothing is described concerning irradiation with ultrasonic waves that are used as thermal energy.

In addition, a method and an apparatus for detecting fat tissues in order to diagnose the distribution of fat tissues, wherein heat is applied to a region of interest through irradiation of light, and a change in the velocity of ultrasonic waves before and after the application of heat is measured so as to detect and define a portion where a change in the velocity of ultrasonic waves has a negative value as fat tissues in order to diagnose visceral fat which is one risk factor of lifestyle related diseases, have been proposed as a new technique for image diagnosis using a change in the velocity of ultrasonic waves before and after the application of heat (Patent Document 2).

The body fat diagnostic apparatus (body fat tissue detecting apparatus) in Patent Document 2 is described below. This apparatus is provided with the main body of the apparatus equipped with a control unit that is necessary to acquire a B mode cross sectional image or an image showing a change in the velocity of ultrasonic waves and a probe that is made to make direct contact with the body surface of a subject so that heat can be applied to the subject or the subject can be irradiated with ultrasonic waves. This probe is a dedicated probe made up of a linear array probe for irradiating the measurement region of a subject with ultrasonic waves and an infrared ray laser source located next to the linear array probe for irradiating the measurement region of the subject with near infrared rays in order to apply heat, where the two are placed side by side laterally so as to target the same measurement region.

The linear array probe has a number of oscillators (formed of piezoelectric elements) that are linearly aligned. Each oscillator sends an ultrasonic wave signal when pulse waves are excited by a drive signal from the control unit, and receives an ultrasonic wave echo signal from the inside of the body of the subject in response to this ultrasonic wave signal. In addition, scanning is made possible by sequentially switching the oscillators for wave transmission and reception in response to a control signal. Furthermore, the infrared ray laser source allows the emission of near infrared rays having a wavelength of 700 nm to 1000 nm from a side of the linear array probe.

The operation of this apparatus for measuring fat in which a change in the velocity of ultrasonic waves is measured is described below. A subject is irradiated with near infrared rays from the infrared laser source so that heat is applied to the subject, and after a predetermined period of time has elapsed, a linear array probe is driven so as to send an ultrasonic wave signal in pulse form for sequential scans, and at the same time, ultrasonic wave echo signals, which are signals received from the subject are sequentially received. In addition, the waveform of the ultrasonic wave signals (reception signals) acquired in a state that the subject is irradiated with the rays is stored as an ultrasonic echo signal after irradiation with rays.

Irradiation with rays is stopped when the storing of the waveform of the received ultrasonic wave signals is completed after irradiation with rays. When a predetermined period of time has elapsed after the stoppage of irradiation and the temperature of the subject has lowered sufficiently, the linear array probe is driven so as to send an ultrasonic wave signal, and the same time, an ultrasonic wave echo signal is received from the subject. Thus, the waveform of the ultrasonic wave signal (reception signal) acquired in a state that irradiation with rays is stopped is stored as an ultrasonic wave echo signal at the time of no irradiation. Here, the stored ultrasonic wave echo signals are displayed as a B mode cross sectional image when the amplitudes thereof are displayed as the brightness.

Next, a change in the velocity of ultrasonic waves is found from the below described relationship on the basis of the ultrasonic wave echo signals after irradiation with rays and at the time of no irradiation.

FIG. 8 is a schematic diagram showing ultrasonic wave signals at the time of no irradiation (before the application of heat) and ultrasonic wave signals after irradiation with rays (after the application of heat) during a certain part of section. The velocity of ultrasonic waves at the time of no irradiation is V, and the velocity of ultrasonic waves after irradiation with rays is V′. In addition, the pulse interval that occurs when the ultrasonic wave signals propagate through a section between certain borders at the time of no irradiation is τ, and the pulse interval that occurs when the ultrasonic wave signals propagate across the same section between the borders (same distance) after irradiation with rays is τ−Δτ. That is to say, the change in the temperature has made the pulse interval shorter by Δτ. Here, the following relationship in Formula (1) is achieved, and thus, a change in the velocity of ultrasonic waves can be calculated from Formula (2) in the following on the basis of the chronological change in the pulse interval between the two echo signals.

V·τ=V′·(τ−Δτ) (1)

V′/V=τ/(τ−Δτ) (2)

Accordingly, the pulse interval (τ) in the region of interest and the amount of the shift in the waveform (Δτ) are calculated from the two echo signals that have been measured, and the change in the velocity of ultrasonic waves (ratio of the change in the velocity of ultrasonic waves (V′/V)) in each portion is calculated from Formula (2).

Next, the portions where the calculated ratio of the change in the velocity of ultrasonic waves (V′/V) has a value smaller than 1 (regions where the ratio of the change in the velocity of ultrasonic waves after the application of heat is negative) are determined as a fat region from among the portions where the ratio of the change in the velocity of ultrasonic waves has been calculated.

That is to say, the velocity of ultrasonic waves that propagate through water is 1524 m/sec, and the velocity of ultrasonic waves that propagate through fat is 1412 m/sec at 37° C.

The changes in the velocity of ultrasonic waves propagating through the two media when the temperature changes are as follows:

Water: +2 m/sec·° C.

Fat: −4 m/sec·° C.

Therefore, the velocity of ultrasonic waves the velocity increases as the temperature of the muscles and organs (the liver and the like) that contain a large amount of water increases, but the velocity of ultrasonic waves decreases in fat portions. Thus, the polarity of the change in the velocity of ultrasonic waves is opposite for these two different media.

Therefore, a fat region can be detected by specifying a region where the change in the velocity of ultrasonic waves has a negative value when the temperature of the measurement region is increased.

In addition, the distribution of the change in the velocity of ultrasonic waves resulting from the analysis is imaged and displayed on a display unit, and thus, fat regions can be clearly distinguished from the other portions in the image.

### PRIOR ART DOCUMENTS

### Patent Documents

Patent Document 1: Japanese Unexamined Patent Publication 2001-145628

Patent Document 2: Japanese Unexamined Patent Publication 2010-005271

### SUMMARY OF THE INVENTION

### 1. Problem to Be Solved by the Invention

The body fat diagnostic apparatus in Patent Document 2 can display an image of a fat region by applying heat to the measurement region and by measuring a change in the velocity of ultrasonic waves using a dedicated probe having a linear array probe and an infrared ray laser source that are aligned side by side.

However, several problems arise as described below in the case of a biometric diagnosis using the above described dedicated probe.

One of the objects for fat diagnosis is the liver (fatty liver). The liver is located inside the ribcage, which is in a deep portion of the body. Therefore, a heat source that can apply heat to the liver is necessary. However, the depth of a portion to which heat can be applied through near infrared rays from an infrared ray laser source is approximately 3 cm to 4 cm from the body surface, and it is theoretically difficult to apply heat to the liver that is located at a depth of 4 cm or greater from the body surface.

In contrast, the depth of a portion to which heat can be applied can be made 4 cm or greater from the body surface depending on the selection of a frequency band in the case where ultrasonic waves are used as thermal energy.

However, ultrasonic waves have other characteristics of being significantly absorbed by bone tissues. For example, the attenuation coefficient of ultrasonic waves is 0.4 to 1.0 (dB/cm) for soft tissues in the body, and is approximately 1.3 to 3.3 (dB/cm) for the muscles, while the attenuation coefficient is approximately 50 (dB/cm) for bone tissues according to a certain report.

Accordingly, in the case where a subject is irradiated with ultrasonic waves instead of light in all directions as in Patent Document 1 by using ultrasonic waves as the thermal energy source, or in the case where the entire body of a subject is laterally irradiated with ultrasonic waves from the ultrasonic wave emitting source of which the width is greater than that of the body of the subject, the ultrasonic wave energy is absorbed by the bone tissues surrounding the target portion, and thus, the bone tissues are thermally damaged, which exposes the subject to danger.

Though a dedicated probe having a linear array probe and an infrared ray laser source that are aligned side by side is used in Patent Document 2, it is possible to attach an ultrasonic wave source for applying heat, instead of the infrared ray laser source, to the linear array probe. In the case where an ultrasonic wave source is simply attached to such a position as to replace the infrared ray laser source, however, the bone tissues in the ribs are irradiated because the ultrasonic wave source for applying heat next to the linear array probe is located directly above the ribs when ultrasound waves for diagnosis are emitted toward the liver from the linear array probe. In this case as well, heat is selectively and intensively applied to the bone tissues and such a problem arises that a subject is exposed to danger.

Therefore, an object of the present invention is to provide a body fat diagnostic apparatus that makes fat diagnosis possible in a deep portion of the body, such as in the liver. Another object is to provide body fat diagnostic apparatus that makes safe diagnosis possible by measuring a change in the velocity of ultrasonic waves even in the case where there are bone tissues outside the measurement region such as in the case of the ribcage when measuring fat in the liver.

### 2. Means for Solving Problem

In order to achieve the above described objects, the present invention provides a body fat diagnostic apparatus with: a probe both that emits ultrasonic waves both for applying heat and for diagnosis; and an ultrasonic wave velocity change analyzing unit for calculating a change in the velocity of ultrasonic waves in a measurement region on the basis of the ultrasonic wave echo signals acquired from the region before and after the application of heat using the above described probe, so that fat diagnosis is conducted on the basis of the calculated change in the velocity of ultrasonic waves.

According to the present invention, ultrasonic waves are used as an energy source for applying heat to the measurement region, and thus, the probe for diagnosis is used also for irradiation through which heat is applied. When the same probe as that for the conventional diagnosis with ultrasonic waves is used, ultrasonic waves can be emitted toward a deep portion in the body through spaces between the ribs in the same manner as in the conventional diagnosis using ultrasonic waves. Therefore, fat diagnosis can be conducted simply by acquiring ultrasonic wave echo signals before and after the application of heat so that the change in the velocity of ultrasonic waves can be calculated.

A switch unit for switching between a signal used for irradiation with ultrasonic waves in order to apply heat and a signal used for irradiation with ultrasonic waves for diagnosis may also be provided.

When a signal used for irradiation with ultrasonic waves in order to apply heat and a signal used for irradiation with ultrasonic waves for diagnosis are switched by means of the switch unit, the ultrasonic wave signal for applying heat and the ultrasonic wave signal for diagnosis can be switched swiftly.

In addition, the switch unit may switch the connection to the above described probe between a terminal on the heat application side that is connected to an ultrasonic wave source for applying heat and a terminal on the diagnosis side that is connected to an ultrasonic wave source for diagnosis.

When the ultrasonic wave source for applying heat and the ultrasonic wave source for diagnosis are provided separately, signals for applying heat and for diagnosis can be independently supplied, and thus, the signal can be stabilized.

In addition, it is preferable for the ultrasonic wave source for applying heat to output continuous waves and for the ultrasonic wave source for diagnosis to output pulse waves.

A high frequency power supply (continuous wave power supplying mechanism) for outputting continuous waves (for example, sine waves) is adopted as the ultrasonic wave source for applying heat so that heat can be easily applied with a sufficient power.

It is preferable for the probe to be made of a number of oscillators that are formed of piezoelectric elements and are linearly aligned.

When a probe in this form is used, it is possible to emit ultrasonic waves towards a deep portion of the body even through a narrow gap between the adjacent ribs. Therefore, heat can be applied to the deep portion of the body while avoiding bone tissues even in the case where continuous ultrasonic waves are emitted for applying heat. General probes are of linear type, sector type or convex type, and any array type where a number of oscillators are aligned including these types may be used as the probe.

In addition, the switch unit may have such a structure that signals are switched so that ultrasonic waves are collectively sent from all of the oscillators for the irradiation with the waves in order to apply heat.

As a result, the measurement region can be efficiently irradiated with ultrasonic waves for applying heat, and thus, uniform heat application is made possible for a short period of time.

At the time of irradiation with ultrasonic waves in order to apply heat, the waves may be sent through a phase shift circuit for shifting the phase in such a manner that the phase of ultrasonic waves from an oscillator at the center of the probe lags relative to that from oscillators at both ends of the probe.

As a result, it is possible to converge ultrasonic waves so that the portion to which heat is to be applied can be intensively irradiated.

In addition, ultrasonic waves used for irradiation for applying heat may be of a frequency band from 1 MHz to 3 MHz.

The depth of a portion from the body surface to which heat can be applied differs depending on the frequency band of ultrasonic waves. When continuous ultrasonic waves have a frequency band of 1 MHz to 3 MHz, heat can be applied even to the liver that is located at a depth of 4 cm or greater from the body surface, which makes fat diagnosis for the liver (diagnosis for a fatty liver) possible due to a change in the velocity of ultrasonic waves.

In addition, ultrasonic waves for diagnosis may have a frequency band higher than that of the ultrasonic waves for applying heat (for example, 1 MHz to 3 MHz).

The frequency band used for image diagnosis in an ultrasonic wave diagnostic apparatus is 1 MHz to 15 MHz, and the higher the frequency of ultrasonic waves is, the higher the resolution of the image is. Meanwhile, the higher the frequency of ultrasonic waves is, the easier ultrasonic waves attenuate within the body of a subject and the shallower the depth of a portion to which heat can be applied is.

In general, the intensity of ultrasonic echo signals required for diagnosis is much smaller than the intensity of ultrasonic waves that need to be inputted for applying heat, and therefore, diagnosis is still possible when the frequency of ultrasonic waves is increased and the ultrasonic wave echo signals from a deep portion are attenuated as outputted.

Thus, the frequency band of ultrasonic waves is set lower so that heat can be applied to a deep portion with the continuous ultrasonic waves for applying heat, and that of the ultrasonic pulse waves for diagnosis is set higher than that in order to increase the resolution of the image. As a result, a satisfactory image showing the change in the velocity of ultrasonic waves having a high image resolution can be gained even for a deep portion of the body.

In addition, ultrasonic waves in the same frequency band (1 MHz to 3 MHz, for example) may be used for ultrasonic waves for applying heat and for ultrasonic waves for diagnosis.

In this case, the frequency band is relatively low for diagnosis, and therefore, the resolution is inferior. However, the cost of the apparatus can be kept low because the bandwidth required for the probe is narrow.

In addition, a B mode signal processing unit for forming a B mode cross sectional image on the basis of the received ultrasonic wave echo signals may be provided.

As a result, a B mode cross sectional image can be checked to determine the measurement point, and thus, fat diagnosis can be conducted for a preferable measurement point.

As for the order in which ultrasonic wave echo signals are acquired before and after the application of heat, the order in which the ultrasonic wave signals before the application of heat are first measured and then the ultrasonic wave signals after the application of heat are measured is preferable from the point of view of time and efforts required for the measurement. However, this order may be altered from the point of the view of the stability of the measurement.

That is to say, it is preferable to first acquire the ultrasonic wave signals immediately after stopping the application of heat as ultrasonic wave echo signals after the application of heat and then to acquire the ultrasonic wave echo signals after the temperature returns back to normal as the ultrasonic wave echo signals before the application of heat.

When heat is applied blood vessels dilate and the blood flow increases in order to prevent a rise in the body temperature. The change in the temperature becomes steep due to an increase in the blood flow, and therefore, the change in the temperature per hour is greater in the measurement when the temperature falls after heat is applied first, and thus, the measurement can be stabilized.

### 3. Effects of the Invention

According to the present invention, ultrasonic waves for diagnosis and ultrasonic waves for applying heat can be emitted from the oscillators in the same probe, and therefore, safe fat measurement is carried out on a deep portion of the body, of which the depth is 4 cm or greater and on a portion inside the ribcage, where fat measurement using a change in the velocity of ultrasonic waves has been difficult according to the prior art.

### DESCRIPTION OF THE DRAWINGS

FIG. 1 is a block diagram showing the entire configuration of the body fat diagnostic apparatus according to one embodiment of the present invention;

FIG. 2 is a diagram showing a portion of the structure for sending and receiving ultrasonic waves for diagnosis and for sending ultrasonic waves for applying heat using a probe in FIG. 1;

FIG. 3 is a flow chart showing the procedure of the measurement operation for the body fat diagnostic apparatus in FIG. 1;

FIG. 4 is a diagram showing another embodiment corresponding to FIG. 2;

FIG. 5 is a block diagram showing the entire configuration of the body fat diagnostic apparatus according to another embodiment of the present invention;

FIG. 6 is a diagram showing a portion of the structure for sending and receiving ultrasonic waves for diagnosis and for sending ultrasonic waves for applying heat using a probe in FIG. 5;

FIG. 7 is a flow chart showing the procedure of the measurement operation for the body fat diagnostic apparatus in FIG. 5; and

FIG. 8 is a schematic diagram showing ultrasonic wave echo signals before and after the application of heat.

### DETAILED DESCRIPTION OF EMBODIMENTS

### First Embodiment

In the following the embodiments of the present invention are described in reference to the drawings.

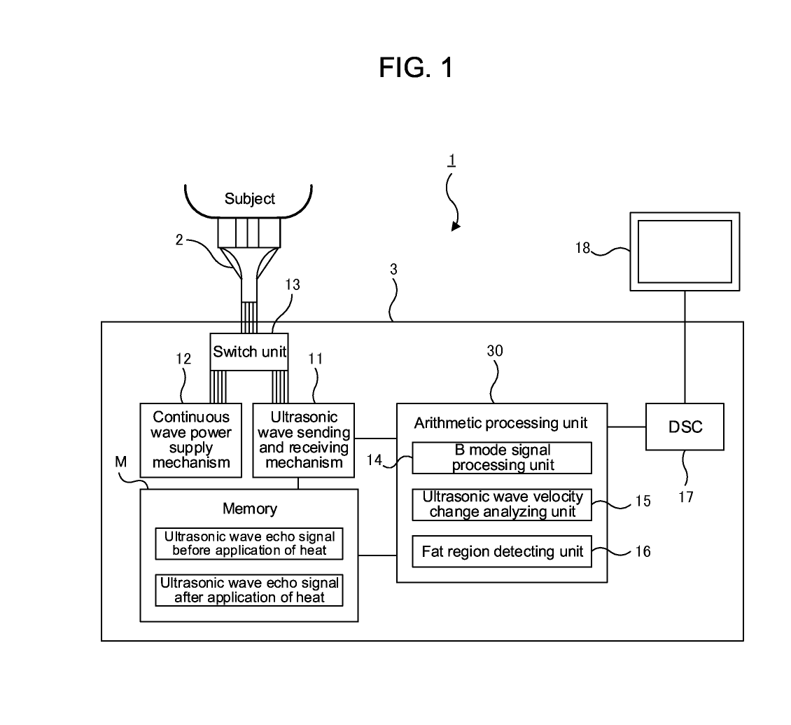

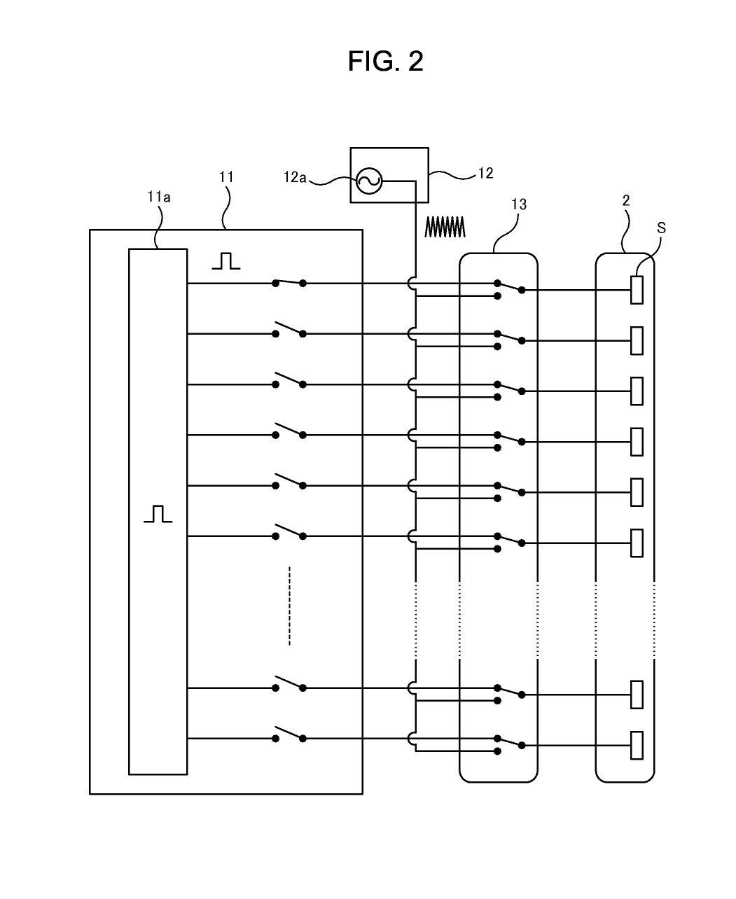

FIG. 1 is a block diagram showing the entire configuration of the body fat diagnostic apparatus according to one embodiment of the present invention, and FIG. 2 is a diagram showing a portion of the structure for sending and receiving ultrasonic waves for diagnosis and for sending continuous ultrasonic waves for applying heat using a probe in FIG. 1.

A body fat diagnostic apparatus 1 is made of a probe 2 and a control unit 3 for controlling the system in order to conduct an ultrasonic wave diagnosis, to apply heat and to conduct a fat diagnosis from the measurement of the change in the velocity of ultrasonic waves, using the probe 2.

The probe 2 is an array type probe (also referred to as an array transducer) where a number of (for example 128) piezoelectric elements that function as oscillators for sending and receiving waves to and from a subject are aligned in a linear form. In order for ultrasonic waves emitted from the oscillators to be able to reach a deep portion through adjacent ribs the thickness of the oscillators is smaller than the gap between the ribs, for example, 15 mm or less. Here, the conventional array type probes for an ultrasonic wave diagnostic apparatus that have been available in the market for the B mode image diagnosis can be used as the probe 2 without change as long as the thickness of the oscillators is appropriate.

The control unit 3 includes a computer having a memory (storage apparatus), a CPU and an input/output apparatus, and generally controls the system that is required for the operation and analysis for a B mode cross sectional image diagnosis and a fat diagnosis. The control unit 3 can be divided into functional blocks for description, then is provided with an ultrasonic wave sending and receiving mechanism 11, a continuous wave power supplying mechanism 12, a switch unit 13, an arithmetic processing unit 30, an image display control unit (digital scan convertor (DSC)) 17 and a display unit 18.

The ultrasonic wave sending and receiving mechanism 11 allows a drive circuit 11a to sequentially drive the system, and thus, controls the scanning by sending ultrasonic pulse waves from the probe 2 as an ultrasonic pulse wave signal for diagnosis so that the ultrasonic pulse waves excite the oscillators S in the probe 2 in a predetermined scanning order. The voltage of the pulse waves that have been sent is approximately 20 V to 60 V and the period of time during which the pulse lasts is approximately 0.5 μs to 5 μs.

Furthermore, the ultrasonic wave sending and receiving mechanism 11 controls the oscillators S so that each oscillator S stands by for and sequentially receives an ultrasonic wave echo signal that is reflected from the subject after an ultrasonic pulse wave signal has been sent. The ultrasonic wave signal received by each oscillator S in the probe 2 is stored in a memory M and at the same time sent to the arithmetic processing unit 30 so that the ultrasonic wave echo signal can be read out for an arithmetic process whenever necessary. In order to find a change in the velocity of ultrasonic waves, the ultrasonic wave sending and receiving mechanism 11 sends an ultrasonic pulse wave signal and receives an ultrasonic wave echo signal twice, before and after the application of heat, and stores the ultrasonic wave echo signal before the application of heat and the ultrasonic wave echo signal after the application of heat, respectively.

The continuous wave power supplying mechanism 12 outputs ultrasonic continuous wave (for example, sine waves) having such a power as to be required for applying heat to a measurement region of a subject from a high frequency power supply 12a and controls the probe 2 so that the oscillators S collectively send waves. The output voltage is approximately 10 V to 20 V, and a power supply dedicated for applying heat is used because a sufficient power for applying continuous waves is necessary. It is known that the depth of a portion in a living body to which heat can be applied is approximately 1/f where f is the frequency of the continuous waves. It is preferable for a portion at a depth of 5 cm or greater form the body surface to be able to be heated in order to diagnosis a fatty liver. The frequency band that makes the heating possible is 1 MHz to 3 MHz.

The switch unit 13 is provided between the oscillators S in the probe 2 and the ultrasonic wave sending and receiving mechanism 11 as well as in between the oscillator S and the continuous wave power supplying mechanism 12. The switch unit 13 is made of an electronic switch or a microrelay, and switches the connection to an oscillator S in the probe 2 between a terminal on the side where the ultrasonic wave sending and receiving mechanism 11 sends an ultrasonic pulse wave signal and receives an ultrasonic wave echo signal while scanning (terminal on the diagnostic side) and a terminal on the side where the continuous wave power supplying mechanism 12 allows the oscillators S to collectively send ultrasonic continuous waves (terminal on the heating side).

The arithmetic processing unit 30 can be divided into functional blocks for description, and is provided with a B mode signal processing unit 14, an ultrasonic wave velocity change analyzing unit 15 and a fat region detecting unit 16.

The B mode signal processing unit 14 carries out a well known B mode cross sectional image forming process on the acquired ultrasonic wave echo signals so as to form a cross sectional image of the beam scanning range, which is then written into the image display control unit (DSC) 17.

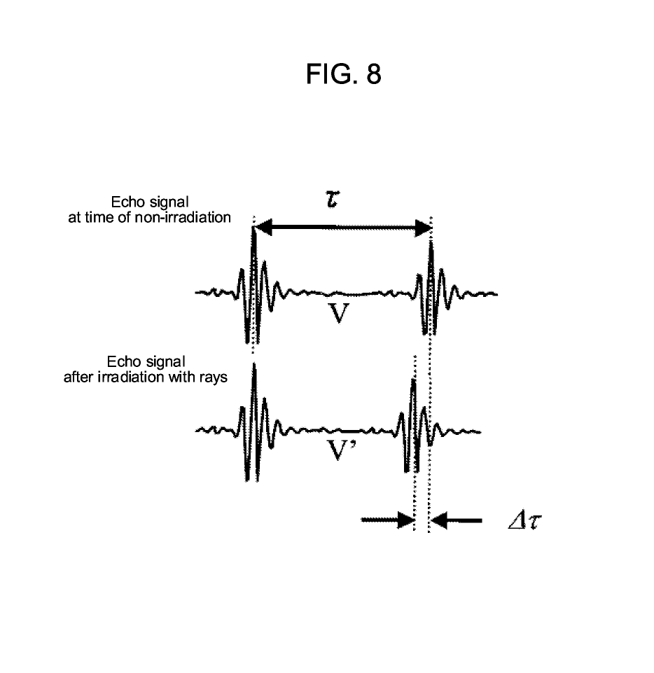

The ultrasonic wave velocity change analyzing unit 15 calculates the amount of the shift of the waveform in the ultrasonic wave echo signal (Δτ) before and after the application of heat on the basis of the ultrasonic wave echo signal received after the application of heat and the ultrasonic wave echo signal received before the application of heat in accordance with the same theory and method as in the prior art in FIG. 8, and also carries out a process for calculating the pulse interval (τ) between borders of tissues within the measurement region. In addition, a process for calculating the ultrasonic wave velocity ratio (V′/V) of each portion from Formula (2) is carried out, and moreover, the ultrasonic wave velocity change image is formed on the basis of the results of the calculations of the ultrasonic wave velocity ratio, and is then written into the image display control unit (DSC) 17.

The fat region detecting unit 16 determines a portion for which the value of the calculated ultrasonic wave velocity ratio (V′/V) is smaller than one to be a fat region, and the data is written into the image display control unit (DSC) 17 so that the fat region image can be displayed on the display unit.

The image display control unit (DSC) 17 controls the system so that the display unit such as a liquid crystal panel displays the image data that has been written into by the arithmetic processing unit 30, for example, a B mode cross sectional image, an ultrasonic velocity change image and a fat region image.

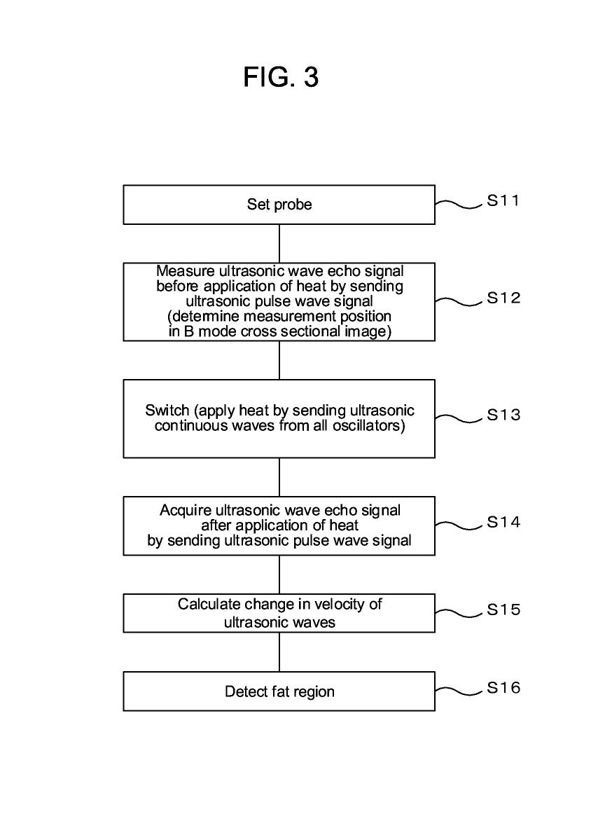

Next, the procedure of the measurement operation in the body fat diagnostic apparatus 1 is described in reference to the flow chart in FIG. 3. Here, diagnosis of a fatty liver is cited as an example for description.

The switch unit 13 is switched to the connection to the “terminal on the diagnostic side” for sending an ultrasonic pulse wave signal (and for receiving an ultrasonic wave echo signal), and the oscillators S in the probe 2 are set so as to target the liver, which is the measurement region, through a gap between ribs of a subject (S11).

Next, the ultrasonic wave echo signal before the application of heat is measured (S12). That is to say, an ultrasonic pulse wave signal is sent, and at the same time, an ultrasonic wave echo signal that is reflected from the subject is received.

At this time, the waves that have been sent and received may be scanned for each oscillator or may be scanned for a number of oscillators that are adjacent to each other so that the measurement regions can be concentrated in a location at a specific depth resulting form a so-called phase superposition. A B mode cross sectional image is formed from the thus acquired ultrasonic wave echo signal before the application of heat so as to be displayed on the display unit.

In addition, the probe 2 is microscopically adjusted if necessary while observing the displayed B mode cross sectional image so that a position that is appropriate for fat measurement is determined and probe 2 is fixed to this position. As a result, the final “ultrasonic wave echo signal before the application heat” is acquired, and the thus acquired data is stored in the memory M.

Next, the probe 2 is kept set to the position where the ultrasonic wave echo signal before the application of heat was acquired, and the switch unit 13 is switched to the connection to the “terminal on the heating side” in order to send ultrasonic continuous waves. Thus, all of the oscillators emit ultrasonic continuous waves so as to apply heat to the measurement region, and the application of heat is maintained until the temperature of the heated region becomes stable (S13). That is to say, heat is applied so that the temperature increase of the subject is in such a range as not negatively affect the subject and as make measurement possible, for example, in a range from approximately 0.5° C. to 2° C. The temperature increase of approximately 2° C. is particularly preferable.

Next, the application of heat is stopped when the temperature is stable in a state where heat is being applied to the measurement region, and at the same time, the switch unit 13 is quickly switched to the connection to the “terminal on the diagnostic side.” In addition, the measurement conditions are made the same as in S12 immediately after the application of heat has been stopped, and an ultrasonic pulse wave signal is again sent and an ultrasonic wave echo signal that is reflected from the subject is received so as to acquire the “ultrasonic wave echo signal after the application of heat,” which is then stored in the memory M (S14).

Here, such a sequence program that a sequential operation can be carried out from the sending of an ultrasonic pulse wave signal immediately after the application of heat has been stopped to the receiving of an ultrasonic wave echo signal simply by carrying out an input operation for stopping the application of heat is build into the apparatus so that the operation thereof can be stabilized.

Next, the ultrasonic wave echo signal after the application of heat and the ultrasonic wave echo signal before the application of heat are read out from the memory M so as to find the pulse interval (τ) and the amount of the shift of the waveform (Δτ), on the basis of which the change in the velocity of ultrasonic waves is calculated through the arithmetic operation using Formula (2) (S15). In addition, an ultrasonic wave velocity change image is formed from the calculated data on the change in the velocity of ultrasonic waves so as to be displayed on the display unit.

Next, a region for which the ultrasonic wave velocity ratio (V′/V) is smaller than one is detected from the calculated data on the change in the velocity of ultrasonic waves, and this region is determined to be a fat region. Then, the area that is determined to be a fat region is displayed on the ultrasonic wave velocity change image (S16).

As a result of the above described operation, an image of the fat region is displayed on the display unit 18, and thus, the results of fat diagnosis are displayed.

The present invention is not limited to the above described embodiment, and various modifications are possible as long as the gist of the present invention is not deviated from.

For example, though the switch unit 13 is provided on the housing side of the control unit 3 in the configuration of the above described embodiment, alternatively a mechanical switch can be provided on the housing side of the probe 2 so that the same switching operation as in FIG. 2 can be achieved, and thus, the switching is made possible with the same hand that is holding the probe 2, which makes the switching operation easy.

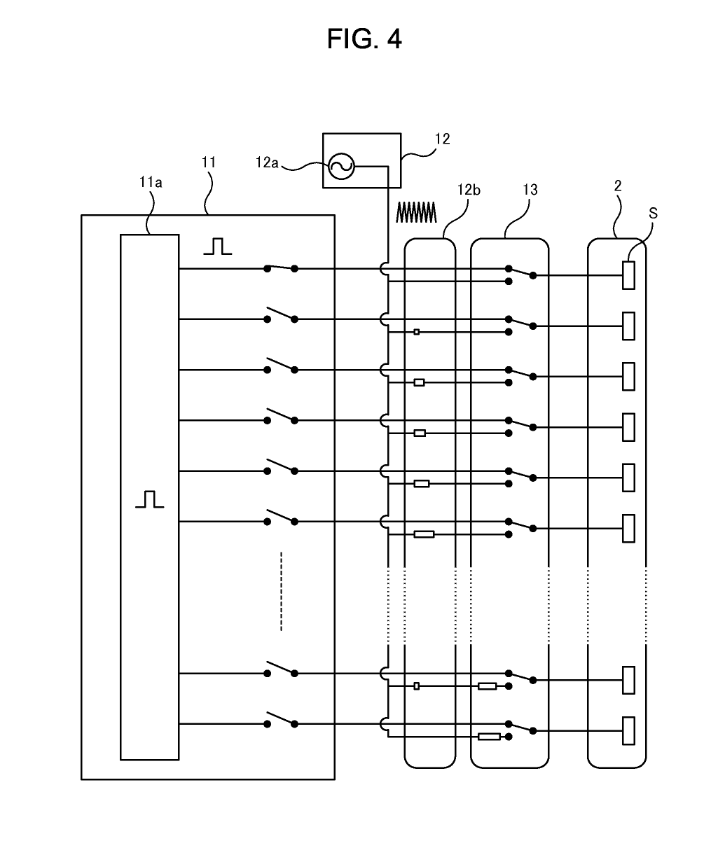

In addition, though ultrasonic continuous waves in the same phase are collectively emitted from the all the oscillators as in FIG. 2 when the switch unit 13 is switched through the connection to the “terminal on the heating side” for sending ultrasonic continuous waves according to the above embodiment, a phase shift circuit 12b may be provided to the terminal on the heating side as shown in FIG. 4. In this phase shift circuit 12b, a signal line to each oscillator S is provided with a phase shifter for shifting the phase of the ultrasonic continuous waves emitted from the oscillator S by a small amount so that the closer an oscillator S is to the center of the probe 2, the larger phase delay it has with respected to the rest of the oscillators, and therefore, the phase advances toward the oscillators on both sides of the probe 2. Thus, the ultrasonic wave beam emitted from the probe 2 becomes a convergent beam having a recess at the center that can apply heat to a subject.

In addition, though an echo signal before the application of heat is acquired first and after that heat is applied through ultrasonic waves and an ultrasonic wave echo signal after the application of the heat is acquired immediately after the application of heat has been stopped when the measurement position is determined by observing the B mode cross sectional image in the above described embodiment, alternatively, heat can be applied first after the measurement position is determined by observing the B mode cross sectional image, and an ultrasonic wave echo signal after the application of heat can be acquired immediately after the application of heat has been stopped after the temperature has reached the desired temperature, and then, an ultrasonic wave echo signal at the time of no application of heat can be acquired in a state that the body temperature has returned to normal, and this may be an echo signal before the application of heat. In this alternative embodiment, though the time and efforts required for measurement increase, blood vessels dilate, which increases the blood flow in order to prevent the body temperature from rising when heat is applied. The change in the temperature becomes steep due to an increase in the blood flow, and therefore, the change in the temperature per hour is greater and the measurement is more stable while the temperature is decreasing than while the temperature is increasing.

### Second Embodiment

Though the ultrasonic wave source for applying heat and the ultrasonic wave source for diagnosis are switched by means of the switch unit 13 in all of the above described embodiments, alternatively, the same ultrasonic wave source may be used for applying heat and for diagnosis.

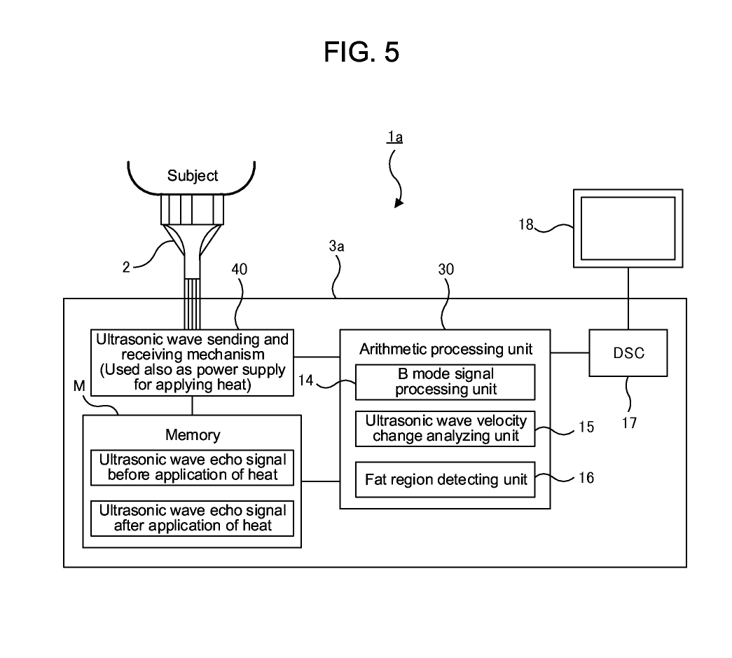

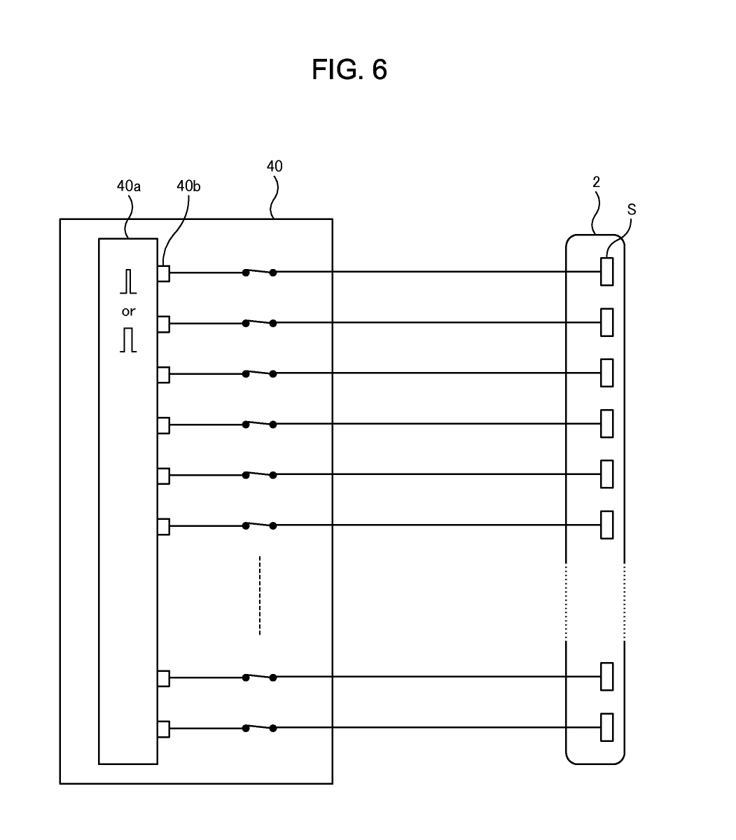

FIG. 5 is block diagram showing the entire configuration of a body fat diagnostic apparatus 1a according to another embodiment of the present invention, and FIG. 6 is a diagram showing a portion of the structure for sending and receiving ultrasonic waves for diagnosis and for sending ultrasonic waves for applying heat using a probe in FIG. 5.

In the control unit 3a in this embodiment, an ultrasonic wave sending and receiving mechanism 40 both for diagnosis and for power supply to apply heat having an improved ultrasonic wave drive circuit (driver) where the output voltage (amplitude of a signal), the waveform (duty cycle ratio of a pulse) and the phase can be greatly changed is used in place of the ultrasonic wave sending and receiving mechanism 11 that is an ultrasonic wave source for diagnosis (see FIG. 1).

That is to say, the drive circuit 40a in the ultrasonic wave sending and receiving mechanism 40 can greatly change the output voltage (amplitude) in addition to the same functions as of the ultrasonic wave sending and receiving mechanism 11 in FIG. 1. For example, the ultrasonic wave sending and receiving mechanism 40 can be used in the range of the output voltage that covers both the output voltage for diagnosis, 20 V to 60 V, and the output voltage for applying heat, 10 V to 20 V.

Furthermore, the waveform of the pulse waves has a short pulse width so that the signal is appropriate for diagnosis, and in addition, the duty cycle ratio can be greatly changed so that the ultrasonic wave signal for the application of heat can be outputted so as to provide such a duty cycle ratio that a pulse wave of 1 MHz to 3 MHz can be regarded as a quasi-continuous wave like a rectangular wave that is close to a continuous wave.

Moreover, a phase shift circuit 40b for changing the phase of the signal that has been sent is provided for each oscillator S in the array type probe 2, and the closer the oscillator is to the center of the probe 2 the larger the phase of the output of the oscillator delays relative to the other oscillators closer to the ends of the probe 2 when waves are collectively sent from the oscillator S when the heat is applied. Here, the phase shift circuit 40b is set so as to be in an off state where no phase shift occurs at the time of diagnosis.

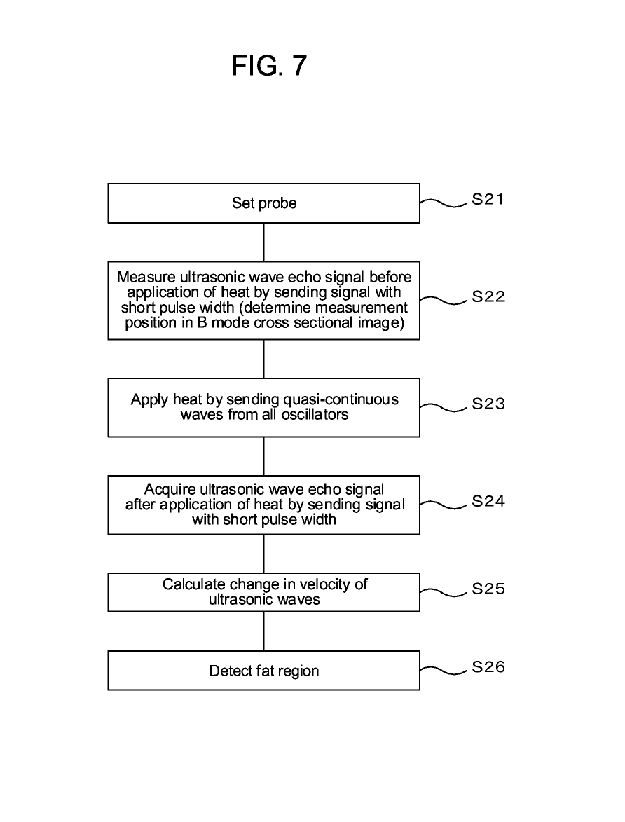

Next, the measurement operation in the body fat diagnostic apparatus 1a is described in reference to the flow chart in FIG. 7. Here, diagnosis of a fatty liver is again cited as an example for description.

The oscillators S in the probe 2 are set so as to target the liver in the measurement region through the gap between ribs of a subject (S21).

Next, an ultrasonic wave echo signal before the application of heat is measured (S22). That is to say, an ultrasonic pulse wave signal having a short pulse wave for diagnosis is sent, and at the same time, an ultrasonic wave echo signal that is reflected from the subject is received.

At this time, the measurement regions may be concentrated in a location at specific depth so that the phases superpose in the same manner as in the first embodiment. A B mode cross sectional image is formed from the thus acquired ultrasonic wave echo signal before the application of heat so as to be displayed on the display unit.

In addition, the probe 2 is microscopically adjusted if necessary while observing the displayed B mode cross sectional image so that a position that is appropriate for fat measurement is determined and probe 2 is fixed to this position. As a result, the final “ultrasonic wave echo signal before the application heat” is acquired, and the thus acquired data is stored in the memory M.

Next, the probe 2 is kept set to the position where the ultrasonic wave echo signal before the application of heat was acquired, and the quasi-continuous waves having a long pulse width for applying heat (having a large duty cycle ratio) are emitted from all of the oscillators so as to apply heat to the measurement region and the application of heat is maintained until the temperature of the heated region becomes stable in a range from approximately 0.5° C. to 2° C. (S23).

Next, the application of heat is stopped when the temperature is stable in a state where heat is being applied to the measurement region, and an ultrasonic pulse wave signal having a short pulse wave for diagnosis is again sent under the same measurement conditions as in S22 immediately after the application of heat has been stopped, and at the same time an ultrasonic wave echo signal that is reflected from the subject is received so as to receive the “ultrasonic wave echo signal after the application of heat” is acquired and stored in the memory M (S24).

Next, the ultrasonic wave echo signal after the application of heat and the ultrasonic wave echo signal before the application of heat are read out from the memory M so as to find the pulse interval (τ) and the amount of the shift of the waveform (Δτ), on the basis of which the change in the velocity of ultrasonic waves is calculated through the arithmetic operation using Formula (2) (S25). In addition, an ultrasonic wave velocity change image is formed from the calculated data on the change in the velocity of ultrasonic waves so as to be displayed on the display unit.

Next, a region for which the ultrasonic wave velocity ratio (V′/V) is smaller than one is detected from the calculated data on the change in the velocity of ultrasonic waves, and this region is determined to be a fat region. Then, the area that is determined to be a fat region is displayed on the ultrasonic wave velocity change image (S26).

As a result of the above described operation, an image of the fat region is displayed on the display unit 18, and thus, the results of fat diagnosis are displayed.

### INDUSTRIAL APPLICABILITY

The present invention can be applied to body fat diagnostic apparatuses for diagnosing fat.

### EXPLANATION OF SYMBOLS

1 body fat diagnostic apparatus
2 linear array type probe
3 control unit
11 ultrasonic wave sending and receiving mechanism
12 continuous wave power supplying mechanism
13 switch unit
14 B mode signal processing unit
15 ultrasonic wave velocity change analyzing unit
16 fat region detecting unit
17 image display control unit (DSC)
18 display unit

What is claimed is:

1. A body fat diagnostic apparatus, comprising:
a probe that emits ultrasonic waves both for applying heat and for diagnosis; and
an ultrasonic wave velocity change analyzing unit for calculating a change in the velocity of ultrasonic waves in a measurement region on the basis of an ultrasonic wave echo signal that has been acquired from the region before and after the application of heat using said probe, wherein
fat diagnosis is conducted on the basis of the calculated change in the velocity of ultrasonic waves.

2. The body fat diagnostic apparatus according to claim 1, further comprising a switch unit for switching between a signal used for irradiation with ultrasonic waves in order to apply heat and a signal used for irradiation with ultrasonic waves for diagnosis.

3. The body fat diagnostic apparatus according to claim 2, wherein said switch unit switches the connection to said probe between a terminal on the heat application side that is connected to an ultrasonic wave source for applying heat and a terminal on the diagnosis side that is connected to an ultrasonic wave source for diagnosis.

4. The body fat diagnostic apparatus according to claim 3, wherein said ultrasonic wave source for applying heat outputs continuous waves and said ultrasonic wave source for diagnosis outputs pulse waves.

5. The body fat diagnostic apparatus according to claim 1, wherein said probe is made of a number of oscillators that are formed of piezoelectric elements and are linearly aligned.

6. The body fat diagnostic apparatus according to claim 5, wherein said switch unit allows connections of the terminal to be switched so that ultrasonic waves are collectively sent from all of the oscillators for the irradiation with the waves in order to apply heat.

7. The body fat diagnostic apparatus according to claim 6, wherein at the time of irradiation with ultrasonic waves in order to apply heat, the waves are sent through a phase shift circuit for shifting the phase in such a manner that the phase of ultrasonic waves from an oscillator at the center of the probe lags relative to that from oscillators at both ends of the probe.

8. The body fat diagnostic apparatus according to claim 1, wherein ultrasonic waves used for irradiation for applying heat are of a frequency band from 1 MHz to 3 MHz.

9. The body fat diagnostic apparatus according to claim 1, wherein ultrasonic waves for diagnosis have a frequency band higher than that of the ultrasonic waves for applying heat.

10. The body fat diagnostic apparatus according to claim 1, wherein ultrasonic waves in the same frequency band are used as ultrasonic waves for applying heat and as ultrasonic waves for diagnosis.

11. The body fat diagnostic apparatus according to claim 1, further comprising a B mode signal processing unit for forming a B mode cross sectional image on the basis of the received ultrasonic wave echo signals.

12. The body fat diagnostic apparatus according to claim 1, wherein the ultrasonic wave signals immediately after stopping the application of heat are first acquired as ultrasonic wave echo signals after the application of heat, and then, the ultrasonic wave echo signals after the temperature returns back to normal are acquired as the ultrasonic wave echo signals before the application of heat.
